SEQUENCE LISTING (23 sequences)

SEQ ID NO: 1 - protein, 189 aa, Mouse
MTEYKLVVVGAGGVGKSALTIQLIQNHFVDEYDPTIEDSYRKQVVIDGETCLLDYLDTAGLEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 2 - DNA, 570 bp, Mouse
atgacagaatacaagcttgtggtggtgggcgctggaggcgtgggaaagagtgccctgaccatccagctgatccagaaccactttgtggacgagtatgatcccactatagaggactcctaccggaaacaggtggtcattgatggggagacatgtctactggactacttagacacagcaggtctagaagagtatagtgccatgcgggaccagtacatgcgcacaggggagggcttcctctgtgtatttgccatcaacaacaccaagtccttcgaggacatccatcagtacagggagcagatcaagcgggtgaaagattcagatgatgtgccaatggtgctggtgggcaacaagtgtgacctggctgctcgcactgttgagtctcggcaggcccaggaccttgctcgcagctatggcatcccctacattgaaacatcagccaagacccggcagggcgtggaggatgccttctatacactagtccgtgagattcggcagcataaattgcggaaactgaacccacccgatgagagtggtcctggctgcatgagctgcaaatgtgtgctgtcctga

SEQ ID NO: 3 - DNA, 30 bp, Artificial Sequence
  5′ primer for knockout allele
gaacctgcgtgcaatccatcttgttcaatg

SEQ ID NO: 4 - DNA, 30 bp, Artificial Sequence
  5′ primer for wild-type allele
actcctcaacatcctggggcagcaacagat

SEQ ID NO: 5 - DNA, 29 bp, Artificial Sequence
  3′ primer
aattgacaagttatgcatccaacagtaca

SEQ ID NO: 6 - DNA, 37 bp, Artificial Sequence
  5′ primer for HRas
tgaattcgccaccatgacagaatacaagcttgtggtg

SEQ ID NO: 7 - DNA, 29 bp, Artificial Sequence
  3′ primer for HRas
actcgagtcaggacagcacacatttgcag

SEQ ID NO: 8 - DNA, 22 bp, Artificial Sequence
  5′ primer for HRasL61
acagcaggtctagaagagtata

SEQ ID NO: 9 - DNA, 22 bp, Artificial Sequence
  3′ primer for HRasL61
tatactcttctagacctgctgt

SEQ ID NO: 10 - DNA, 24 bp, Artificial Sequence
  5′ primer for hras
atgacagaatacaagcttgtggtg

SEQ ID NO: 11 - DNA, 22 bp, Artificial Sequence
  3′ primer for hras
attaaccctcactaaagggaag

SEQ ID NO: 12 - DNA, 21 bp, Artificial Sequence
  5′ primer for s100a6
atggcatgccctctggatcag

SEQ ID NO: 13 - DNA, 25 bp, Artificial Sequence
  3′ primer for s100a6
ttatttcagagcttcattgtagatc

SEQ ID NO: 14 - DNA, 24 bp, Artificial Sequence
  5′ primer for prom1
aggctactttgaacattatctgca

SEQ ID NO: 15 - DNA, 21 bp, Artificial Sequence
  3′ primer for prom1
ggcttgtcataacaggattgt

SEQ ID NO: 16 - DNA, 20 bp, Artificial Sequence
  5′ primer for contactin
gtcaccagccaggagtactc

SEQ ID NO: 17 - DNA, 20 bp, Artificial Sequence
  3′ primer for contactin
caggagcaagctgaggagac

SEQ ID NO: 18 - DNA, 22 bp, Artificial Sequence
  5′ primer for mbp
atggcatcacagaagagaccct

SEQ ID NO: 19 - DNA, 23 bp, Artificial Sequence
  3′ primer for mbp
ctgtctcttcctcccagcttaaa

SEQ ID NO: 20 - DNA, 21 bp, Artificial Sequence
  5′ primer for plp
gacaagtttgtgggcatcacc

SEQ ID NO: 21 - DNA, 21 bp, Artificial Sequence
  3′ primer for plp
tcggcccatgagtttaaggac

SEQ ID NO: 22 - DNA, 21 bp, Artificial Sequence
  5′ primer for sox8
ctatggaggcgcttcctactc

SEQ ID NO: 23 - DNA, 20 bp, Artificial Sequence
  3′ primer for sox8
acaggctggtcccagttgct

(19) United States
(12) Patent Application Publication
(10) Pub. No.: US 2008/0293056 A1
(43) Pub. Date: Nov. 27, 2008

(54) METHOD FOR PREPARING CANCER STEM CELLS

(75) Inventor: Toru KONDO, Kobe-shi (JP)

Correspondence Address: Studebaker & Brackett PC, 1890 Preston White Drive, Suite 105, Reston, VA, 20191, US

(73) Assignee: RIKEN, Wako-shi (JP)

(21) Appl. No.: 12/056,975

(22) Filed: Mar. 27, 2008

(30) Foreign Application Priority Data
Apr. 18, 2007 (JP) 2007-109539

(51) Int. Cl. C12Q 1/68; C12N 15/87; C12N 5/06; C12Q 1/02

(52) U.S. Cl. 435/6; 435/455; 435/366; 435/29

(57) ABSTRACT

The present invention provides a method for preparing cancer stem cells including the step of subjecting normal cells to Ras activation and p53 deficiency; the cancer stem cells prepared by the preparation method; a method for screening a cancer stem cell-targeting substance and a method for screening an anti-cancer substance using the cancer stem cells; a method for treating a cancer comprising administering to a patient the substances obtainable by the screening methods; and a diagnostic method for cancers including the step of detecting proteins specifically expressed in the cancer stem cells or mRNAs of the protein.

### CROSS-REFERENCE TO RELATED APPLICATION

This application claims the priority to Japanese Patent Application No. 2007-109539, which is incorporated herein by reference in their entirety.

### BACKGROUND OF THE INVENTION

The present invention relates to a method for preparing cancer stem cells, the cancer stem cells prepared by the preparation method, a method for screening a cancer stem cell-targeting substance and a method for screening an anti-cancer substance using the cancer stem cells, a method for treating cancers comprising a step of administering to a patient the substances obtainable by the screening methods, and a diagnostic method for cancers including the step of detecting proteins or mRNAs thereof specifically expressed in the cancer stem cells.

In principle, cancer stem cells (CSCs) may originate from normal tissue stem cells or in progenitor cells or differentiated cells that have acquired features of the stem cell by oncogenic mutation. The CSCs continuously generate proliferating cancer cells for forming most of cells in a tumor by self-regeneration (Reya, T., Morrison, S. J., Clarke, M. F. & Weissman, I. L. Stem cells, cancer, and cancer stem cells. Nature 414, 105-111 (2001)). While it has been shown that mutations of some oncogenes and cancer repressor genes are involved in tumorigenesis, the relation between source cells of the CSCs and changes of the genes has not been elucidated yet except in certain kinds of leukemia (Cozzio, A. et al. Similar MLL-associated leukemias arising from self-renewing stem cells and short-lived myeloid progenitors. Genes Dev. 17, 3029-3035 (2003); Huntly, B. J. et al. MOZ-TIF2, but not BCR-ABL, confers properties of leukemic stem cells to committed murine hematopoietic progenitors. Cancer Cell 6, 587-596 (2004); Krivtsov, A. V. et al. Transformation from committed progenitor to leukaemia stem cell initiated by MLL-AF9. Nature 442, 818-822 (2006); and Somervaille, T. C. & Cleary, M. L. Identification and characterization of leukemia stem cells in murine MLL-AF9 acute myeloid leukemia. Cancer Cell 10, 257-268 (2006)). The present inventors and other researchers have shown that specified oligodendrocyte progenitor cells (OPCs) and astrocytes (ASTs) could be restored to their neural stem cell-like cells by cultivating the cells under an appropriate condition (Kondo, T. & Raff, M. Oligodendrocyte precursor cells reprogrammed to become multipoteintial CNS stem cells. Science 289, 1754-1757 (2000); Laywell, E. D. et al. Identification of a multipotent astrocytic stem cell in the immature and adult mouse brain. Proc. Natl. Acad. Sci. USA 97, 13889-13894 (2000); Belachew, S. et al. Postnatal NG2 proteoglycan-expressing progenitor cells are intrinsically multipotent and generate functional neurons. J. Cell Biol. 161, 169-186 (2003); and Nunes, M. C. et al. Identification and isolation of multipotential neural progenitor cells from the subcortical white matter of the adult human brain. Nature Med. 9, 439-447 (2003)). These facts suggest that the OPCs and ASTs, and neural stem cells (NSCs) might be the source cells of cerebral CSCs. P53 is a tumor suppressor gene that most frequently mutates in human cancers including human gliomas (Rasheed, B. K. et al. Alterations of the TP53 gene in human gliomas. Cancer Res. 54, 1324-1330 (1994)). Elevation of Ras activity has also been reported in human gliomas and glioma cell strains (Guha, A. et al. Proliferation of human malignant astrocytomas is dependent on Ras activation. Oncogene 15, 2755-2765 (1997)). It has been further shown that a combination of Ras activation and p53 deficiency in ASTs may cause malignant gliomas in mice (Reilly, K. M. et al. Nf1; Trp53 mutant mice develop glioblastoma with evidence of strain-specific effects. Nature Genet. 26, 109-113 (2000); Zhu, Y. et al. Early inactivation of p53 tumor suppressor gene cooperating with NF1 loss induces malignant astrocytoma. Cancer Cell 8, 119-130 (2005); Bizub, D., Blair, D., Alvord, G. & Skalka, A. M. Correlation between H-ras p21TLeu61 protein content and tumorigenicity of NIH3T3 cells. Oncogene 3, 443-448 (1988); and Barnett, S. C. & Crouch, D. H. The effect of oncogenes on the growth and differentiation of oligodendrocyte type 2 astrocyte progenitor cells. Cell Growth Differ. 6, 69-80 (1995)). However, it has not been known whether the combination could induce transformation of cultured NSCs, OPCs and ASTs into CSCs.

Developments in recent years as described above have shown that malignant tumors contain the CSC having a tumorigenic ability by infinite auto-replication. While it has been recently shown that leukemia stem cells may be derived from either hematopoietic stem cells or restrictive progenitor cells (Cozzio, A. et al. Similar MLL-associated leukemias arising from self-renewing stem cells and short-lived myeloid progenitors. Genes Dev. 17, 3029-3035 (2003); Huntly, B. J. et al. MOZ-TIF2, but not BCR-ABL, confers properties of leukemic stem cells to committed murine hematopoietic progenitors. Cancer Cell 6, 587-596 (2004); Krivtsov, A. V. et al. Transformation from committed progenitor to leukaemia stem cell initiated by MLL-AF9. Nature 442, 818-822 (2006); and Somervaille, T. C. & Cleary, M. L. Identification and characterization of leukemia stem cells in murine MLL-AF9 acute myeloid leukemia. Cancer Cell 10, 257-268 (2006)), it is not clear whether the CSC in solid cancers is derived from tissue-specific stem cells, restrictive progenitor cells or differentiated cells. For example, Krivtsov, A. V. et al. Transformation from committed progenitor to leukaemia stem cell initiated by MLL-AF9. Nature 442, 818-822 (2006) and Somervaille, T. C. & Cleary, M. L. Identification and characterization of leukemia stem cells in murine MLL-AF9 acute myeloid leukemia. Cancer Cell 10, 257-268 (2006) describe that leukemia stem cells may be obtainable by introducing oncogene MLL-AF9 into normal progenitor cells. On the other hand, the presence of CSCs is only reported in cerebral tumors and mammary cancers in the study of CSCs in solid cancers (Singh, S. K. et al. Identification of human brain tumour initiating cells. Nature 432, 396-401 (2004); Galli, R. et al. Isolation and characterization of tumorigenic, stem-like neural precursors from human glioblastoma. Cancer Res 64, 7011-7021 (2004); Bao, S. et al. Glioma stem cells promote radioresistance by preferential activation of the DNA damage response. Nature 444, 756-760 (2006); Piccirillo, S. G. et al. Bone morphogenetic proteins inhibit the tumorigenic potential of human brain tumour-initiating cells. Nature 444, 761-765 (2006); and Hambardzumyan, D., Squatrito, M. & Holland, E. C. Radiation resistance and stem-like cells in brain tumors. Cancer Cell 10, 454-456 (2006)). Since the source cells of the CSCs and oncogenes involved in transformation of the source cells into the CSCs have not been sufficiently studied, it has been desired to obtain more knowledge for developing effective therapeutic methods of cancers.

### SUMMARY OF THE INVENTION

An object of the present invention is to provide a method for preparing cancer stem cells, the cancer stem cells prepared by the preparation method, a method for screening a cancer stem cell-targeting substance and a method for screening an anti-cancer substance using the cancer stem cells, a method for treating cancers comprising a step of administrating to a patient the substances obtainable by the screening methods, and a diagnostic method for cancers including the step of detecting proteins or mRNAs thereof specifically expressed in the cancer stem cells.

The present inventors have engaged in the above-mentioned object on gliomas by simultaneously causing activation of a Ras signal pathway and suppression of p53 (this frequently occurs in human gliomas (Louis, D. N. The p53 gene and protein in human brain tumors. J. Neuropathol. Exp. Neurol. 53, 11-21 (1994); Bogler, O., Huang, H. J., Kleihues, P. & Cavenee, W. K. The p53 gene and its role in human brain tumors. Glia 15, 308-327 (1995); Feldkamp, M. M., Lau, N. & Guha, A. Signal transduction pathways and their relevance in human astrocytomas. J. Neurooncol. 35, 223-248 (1997); Ohgaki, H. et al. Genetic pathways to glioblastoma: a population-based study. Cancer Res. 64, 6892-6899 (2004); and Hulleman, E. & Helin, K. Molecular mechanisms in gliomagenesis. Adv. Cancer Res. 94, 1-27 (2005)) in oligodendrocyte progenitor cells (OPCs) and differentiated astrocytes (ASTs). The present inventors have shown that, while both the OPCs and the NSCs are transformed into glioma stem cells accompanying with re-programming of a wide range of gene expression under these conditions, the ASTs are not transformed. While all of the three cell types showed enhanced proliferation, only the NSCc and OPCs formed glioblasts upon transplantation of the cells into the brain of a nude mouse. These findings suggest the possibility that the glioblast may be derived from either the OPCs or the NSCs in humans. The present invention has been completed based on these lines of findings.

The present invention provides:

(1) A method for preparing cancer stem cells, comprising the step of subjecting normal cells to Ras activation and p53 deficiency;

(2) The method according to (1), wherein the cancer stem cells are glioma stem cells;

(3) The method according to (1), wherein the normal cells are neuronal stem cells or oligodendrocyte progenitor cells;

(4) The method according to (1), wherein Ras activation is achieved by introduction of an HRas^{L61} gene;

(5) The method according to (1), wherein p53 deficiency is achieved by using cells originated from a p53 deficient animal;

(6) Cancer stem cells obtainable by the method according to any one of claims 1 to 5.

(7) A method for screening a cancer stem cell-targeting substance, comprising the step of identifying the cancer stem cell-targeting substance by determining molecules specifically expressed in the cancer stem cells according to (6);

(8) A method for treating cancer in a patient, comprising the step of administering to said patient the cancer stem cell-targeting substance obtainable by the method according to (7);

(9) A method for screening an anti-cancer substance, comprising the step of adding a candidate substance to the cancer stem cells according to (6);

(10) A method for treating cancer in a patient, comprising the step of administering to said patient the cancer stem cell-targeting substance obtainable by the method according to (9); and

(11) A method for diagnosing cancers, comprising the step of detecting proteins or mRNAs thereof specifically expressed in the cancer stem cells according to (6).

Accordingly, the present invention provides a method for preparing cancer stem cells, the cancer stem cells prepared by the preparation method, a method for screening a cancer stem cell-targeting substance and a method for screening an anti-cancer substance using the cancer stem cells, pharmaceutical compositions containing the substances obtainable by the screening methods, and a diagnostic method for cancers including the step of detecting proteins specifically expressed in the cancer stem cells or mRNAs of the protein.

### BRIEF DESCRIPTION OF THE DRAWINGS

FIG. 1A shows characterization of NSCs, ASTs and OPCs deficient in HRas^{L61} transfect p53, wherein the p53 deficient NSC, AST and OPC (a) were immunolabeled with nerve series markers and DAPI (diaminediphenyl indole), scale bar=15 μm, and wherein the proportions of the nerve series marker-positive cells in NSC, AST and OPC as non-transfected cells (b), NSC-C, AST-C and OPC-C as control vector transfected cells (c), and NSC-L61, AST-L61 and OPC-L61 as HRas^{L61} vector transfected cells (d) are shown by an average value ±SD in three times of cultivation;

FIG. 1B shows characterization of the HRas^{L61} transfect p53-deficient NSC, AST and OPC;

FIG. 2 shows that the brain CSC is enriched in NSC-L61 and OPC-L61 but not in AST-L61, wherein (a) shows proliferation of both a non-transfected cell (∘) and an HRas^{L61} vector transfected cell () was measured by a WST-1 assay, and the results are shown by an average value ±SD in three times of cultivation, and (b) shows survival curves of the mice infected with solutions of limiting dilution of NSC-L61 (left panel), OPC-L61 (center panel) and AST-L61 (right panel) (the experiments were repeated at least twice and the same results were obtained);

FIG. 3A shows pathological features of the brain tumor induced from NSC-L61 and OPC-L61, wherein (a) to (d) and (e) to (h) show slices of brains having tumors induced from NSC-L61 and slices of brains having tumors induced from OPC-L61 ((a) to (h)), respectively, where both tumors show infiltration into the parenchyma ((a) and (e)), necrosis (arrows in (b) and (f)), hypercellularity and hypervascularity ((c) and (g)), multinucleated giant cells (white arrows in (d) and (h)) and mitotic cells (solid arrows in (d) and (h)); these pathological features resemble those of human GBM (magnification: 40 ((b) and (f)), 200 ((c) and (g)), and 400 ((d) and (h)); and (i) the proportion of nerve series marker-positive cells in the tumor is expressed as an average value ±SD in three times of cultivation;

FIG. 3B shows pathological features of brain tumors induced from NSC-L61 and OPC-L61;

FIG. 4A shows a line of evidence of collective changes of gene expression in OPC-L61, wherein (a) hierarchical clustering using Affymetrix Murine 430A 2.0 microarray shows that OPC-L61 shows a gene expression profile closest to NSC-L61; (b) 60 probe sets of higher ranking are shown for the genes that show increased expression in NSC, NSC-L61, OPC and OPC-L61; (c) expression of NSC and OPC-specific genes are analyzed by RT-PCR in NSC, NSC-L61, OPC and OPC-L61; and (d) immunoreactivity of NSC, NSC-L61, OPC and OPC-L61 on prominin-1 is shown by an FACS analysis, where the same results were obtainable by repeating three times of experiments;

FIG. 4B shows the evidence of collective changes of expression of genes in OPC-L61;

FIG. 4C shows the evidence of collective changes of expression of genes in OPC-L61;

FIG. 4D shows the evidence of collective changes of expression of genes in OPC-L61;

FIG. 5A shows immunoreactivity of the HRas^{L61} transfect p53-deficient NSC, wherein the NSC was transfected with a control vector (a) or an HRas^{L61} expression vector (b) followed by cultivation for 2 to 3 weeks and immunological labeling with nerve series markers and GFP, and cell nuclei were stained with DAPI (scale bar=15 μm);

FIG. 5B shows immunoreactivity of the HRas^{L61} transfect p53-deficient NCS;

FIG. 6A shows immunoreactivity of the HRas^{L61} transfect p53-deficient AST, wherein the AST was transfected with a control vector (a) or an HRas^{L61} expression vector (b) followed by cultivation for 2 to 3 weeks and immunological labeling with neural series markers and GFP, and cell nuclei were stained with DAPI (scale bar=15 μm);

FIG. 6B shows immunoreactivity of the HRas^{L61} transfect p53-deficient AST;

FIG. 7A shows immunoreactivity of the HRas^{L61} transfect p53-deficient OPC, wherein OPC was transfected with a control vector (a) or an HRas^{L61} expression vector (b) followed by cultivation for 2 to 3 weeks and immunological labeling with neural series markers and GFP, and cell nuclei were stained with DAPI (scale bar=15 μm);

FIG. 7B shows immunoreactivity of the HRas^{L61} transfect p53-deficient OPC;

FIG. 8 shows a colony-forming assay of the HRas^{L61} transfect nerve cells on soft agar, wherein NSC, AST and OPC were transfected with a control vector or an HRas^{L61} expression vector followed by cultivation in soft agar for 20 days, and the colony was stained with crystal violet; and

FIG. 9 shows that secondary brain tumors quite resembles to primary tumors, wherein primary tumors formed by NSC-L61 (a) and OPC-L61 (b), respectively, were isolated, and 1,000 cells of the GFP-positive cells were transplanted to the second mouse brain, where H & E staining of the secondary tumor (right panel) shows features of human GBM (including necrosis, hypercellularity and hypervascularity, multinucleated giant cells (white arrow) and mitotic cells (solid arrow)), which resembles to the primary tumor (left panel) (magnification: 200).

### DETAILED DESCRIPTION OF THE PREFERRED EMBODIMENTS

The present invention provides a method for preparing cancer stem cells including the step of subjecting normal cells to Ras activation and p53 deficiency, and the cancer stem cells prepared by the preparation method.

The stem cell as used herein refers to a cell having a self-regenerative ability. Of the stem cells, the cells having an ability to differentiate into normal cells are referred to as normal stem cells, while the cells having an ability to differentiate into cancer cells are referred to as cancer stem cells. The cancer stem cells include those that may be differentiated into arbitrary cancer cells. The cancer is a solid cancer, preferably a glioma, for example.

The self-regenerative ability of the cancer stem cell may be confirmed by a continuous transplantation experiment by which, for example, a tumor formed by transplantation of the cancer stem cells is excised from an animal, followed by investigating whether the same tumor is formed or not when re-transplanting the tumor cells to another animal.

The cell population containing the cancer stem cells obtainable by the preparation method of the present invention has an ability for forming a tumor with, for example, 100 cells, preferably 50 cells, more preferably 20 cells and further preferably 10 cells upon transplantation to a nude mouse. Alternatively, the cell population containing the cancer stem cells obtainable by the preparation method of the present invention contains, for example, 1 cancer stem cell in 100 cells, preferably in 50 cells, more preferably in 20 cells and further preferably in 10 cells.

The cancer stem cell that causes a glioma is referred to as a glioma stem cell. The glioma is a common name of tumors derived from neuroectodermal tissues of cerebral parenchyma, and includes astrocytoma, polymorphic glioblastoma (GBM), medulloblastoma, ependymoma, oligodendroglioma and choroidal papilloma. The GBM is the most malignant glioma among them, and is featured by hypercellularity, polymorphism, multinucleated giant cells, mitosis, necrosis and infiltration.

Ras activation as used herein refers to activation of a Ras signal pathway. The Ras protein includes H-Ras, K-Ras and N-Ras. The normal Ras protein has a GTP/GDP-binding GTPase activity. Ras is activated by binding to GTP to enable signal transmission. On the other hand, GTP is inactivated by being hydrolyzed to GDP due to Ras's own GTPase activity. For example, Ras exhibits a transforming activity by being put into a steadily activated state (i.e. becomes oncogenic) when the GTPase activity is lost due to mutation of the Ras gene that encodes the Ras protein. Examples of mutation in the ras gene that exhibits the transforming activity include substitutions of 12th, 13th and 61st amino acids. Any methods may be used for the purpose of the present invention so long as Ras is activated by the above-mentioned activation methods. For example, a gene that encodes H-Ras (HRas^{L61}) having a mutation for substituting 61st glutamine with leucine (Bizub, D., Blair, D., Alvord, G. & Skalka, A. M. Correlation between H-ras p21TLeu61 protein content and tumorigenicity of NIH3T3 cells. Oncogene 3, 443-448 (1988); and Barnett, S. C. & Crouch, D. H. The effect of oncogenes on the growth and differentiation of oligodendrocyte type 2 astrocyte progenitor cells. Cell Growth Differ. 6, 69-80 (1995)) is integrated into an arbitrary expression vector, thereby Ras may be activated. The amino acid sequence of HRas^{L61} and the nucleotide sequence of the gene that encodes HRas^{L61} are shown by SEQ. ID NO.: 1 and SEQ. ID NO.: 2, respectively. A codon CAA that encodes 61st amino acid (glutamine; Gln) of H-Ras is substituted with CTA (positions 181 to 183 of SEQ. ID NO.: 2), and the amino acid encoded by the codon CTA corresponds to leucine (Leu: position 61 of SEQ. ID NO.: 1).

P53 deficiency as used herein refers to a defect or decrease in the action of p53 protein. The normal p53 gene serves as a cancer suppressing gene, while an abnormality in p53 gene is considered to lead to proliferation and oncogenesis of the cells. Any methods may be used for the purpose of the present invention so long as the above-mentioned deficiency is achieved. Specifically, p53 deficiency may be achieved by mutation of p53 gene itself that encodes p53 protein as well as by mutations of a gene that regulates expression of p53 gene and of a gene that is regulated by the p53 protein. Known techniques such as destruction of the gene and RNAi may be used for this purpose. For example, p53 deficiency may be achieved by using cells derived from p53 deficient animals such as an E14.5p53 (−/−) mouse (Tsukada, T., et al. Enhanced proliferative potential in culture of cells from p53-deficient mice. Oncogene 8, 3313-3322 (1993)).

The normal cell as used herein refers to a normal (i.e. not oncogenic) cell. The normal cell is preferably a restrictive cell, more preferably a nervous system cell. Examples of the normal nervous system cell include a neural stem cell (NSC), an oligodendrocyte progenitor cell (OPC), an astrocyte (AST) and an oligodendrocyte. The neural stem cell (NSC) and the oligodendrocyte progenitor cell (OPC) are preferably used.

The present invention provides a method for screening a cancer stem cell-targeting substance including the step of determining a molecule that is specifically expressed in the cancer stem cell for identifying the cancer stem cell-targeting substance, and a method for screening an anti-cancer substance including the step of adding a candidate substance to the cancer stem cell. In addition, the present invention provides a method for treating cancers in a patient, comprising a step of administrating to said patient the cancer stem cell-targeting substance or the anti-cancer substance obtainable by the above-mentioned methods.

In conventional therapeutic methods of cancers such as chemotherapy, a small number of the cancer stem cells in non-cancer stem cells having no tumor-forming ability may survive and the survived cancer cells may result in recurrence of the cancer. If a cancer stem cell-targeting substance could be identified by determining a molecule that is specifically expressed in the cancer stem cell having a tumor-forming ability, a therapeutic method of cancers that targets the cancer stem cell may be developed by using the molecule. The cancer stem cell-targeting substances available include antibodies or fragments thereof, or corresponding receptors and ligands. An anti-cancer agent that acts on the cancer stem cell and is more effective than conventional ones may be developed if a substance that acts on the cancer stem cell could be identified by adding candidate substances (for example, a low-molecular drug) to the cancer stem cell. Such screening is possible according to the preparation method of the present invention since a large quantity of the cancer stem cells may be readily obtained in a high purity as compared with the conventional condensation methods using surface markers.

As mentioned above, in the method for treating a cancer of the present invention, the cancer stem cell-targeting substance or the anti-cancer substance obtainable by the above-mentioned methods can be administered to a patient. In addition, conventionally used anti-cancer drugs may be used together. In general, these ingredients may be administrated in a pharmaceutical composition with other components such as an optional carrier generally used in the pharmaceutical composition. The route of administration and dosage may be appropriately determined by those skilled in the art.

In the method for treating cancers of the present invention, the cancer stem cell-targeting substance or the anti-cancer substance obtainable by the above-mentioned method is used. Additionally, conventionally used anti-cancer drugs may be used in the method for treating cancers of the present invention.

The present invention provides a diagnostic method of a cancer including the step of detecting a protein specifically expressed in the cancer stem cell or an mRNA of the protein. Such proteins may be identified, for example, by using an antibody against various surface markers. These mRNAs may be identified, for example, by using a DNA microarray. These methods are publicly known in the art. The cancer stem cell having a self-regenerative ability may be specifically detected by detecting the protein specifically expressed in the cancer stem cell or an mRNA of the protein, thereby enabling more detailed diagnosis of a cancer. An antibody or fragments thereof, or corresponding receptors or ligands may be used for detecting the protein. Hybridization with a nucleic acid probe, amplification of the nucleic acid using a nucleic acid amplification method or a combination thereof may be used for detecting the mRNA.

While the present invention is described in more detail with reference to examples, the scope of the present invention is by no means restricted by these examples.

### Animal and Chemical Substances

The animals were obtained from the Mutant Mouse Development Team, Center for Developmental Biology (CDB), and NIPPON SLC Co. The genotype was analyzed by PCR. The following oligonucleotide DNA primers were synthesized: 5′-primer for knockout allele (5′-GAACCTGCGTGCAATCCATCTTGTTCAATG-3′: SEQ. ID NO.: 3), 5′-primer for wild type allele (5′-ACTCCTCAACATCCTGGGGCAGCAACAGAT-3′: SEQ. ID NO.: 4); and 3′-primer (5′-AATTGACAAGTTATGCATCCAACAGTACA-3′; SEQ. ID NO.: 5). The cycle parameters were; 94° C. for 45 seconds, 60° C. for 1 minute and 72° C. for 3 minutes, and the cycle was repeated 30 times (Tsukada, T., et al. Enhanced proliferative potential in culture of cells from p53-deficient mice. Oncogene 8, 3313-3322 (1993)). Chemical substances and growth factors were purchased from Sigma Co. and PeproTech Co. except those individually shown.

### Cell Culture

NSCs were prepared from the telencephalon of a p53 deficient mouse on the embryonic day 13.5, and was expanded in bFGF (10 mg/mL) and EGF (10 mg/mL) (Kondo, T. & Raff, M. Chromatin remodeling and histone modification in the conversion of oligodendrocyte precursors to neural stem cells. Genes Dev. 18, 2963-2972 (2004)). The spheres were cultivated for one day for immunostaining on an 8-well chamber slide (Nunc Co.) coated with poly-D-lysine (PDL, 15 μg/mL) and fibronectin (1 μg/mL, Invitrogen Co.) in the presence of bFGF. ASTs were induced from NSCs by cultivation in DMEM containing 10% FCS for 3 weeks. OPCs were induced from NSCs by cultivation in a serum-free Dulbecco's modified Eagle's medium (OPC medium) containing bovine insulin (10 mg/mL), human transferrin (100 mg/mL), BSA (100 mg/mL), progesterone (60 ng/mL), putrescine (16 mg/mL), sodium selenite (40 ng/mL), N-acetyl cysteine (60 mg/mL), forskolin (5 mM), PDGFAA (10 ng/mL), bFGF (2 ng/mL), 0.25% FCS, penicillin and streptomycin (GIBCO Co.). The induced OPCs were expanded, and purified by sequential immunopanning (Wang, S. et al. A role for the helix-loop-helix protein Id2 in the control of oligodendrocyte development. Neuron 29, 603-614 (2001)).

### Transfection

Transfections into NSCS, ASTs and OPCs were implemented using the Nucleofector protocol according to the instruction manual by the manufacturer (AMAXA). Briefly, 2×10⁶ cells were suspended in a Mouse NSC Nucleofector Solution (100 μL) containing 10 μg of either a pCMS-EGFP control vector (Clontech Co.) or pCMS-EGFP-HRas^{L61}, and the cells were transfected with the vector using a Nucleofector device. The transfected cells were cultivated in respective optimum media.

### Immunostaining

Cells were subjected to immunostaining as described before (Kondo, T. & Raff, M. Chromatin remodeling and histone modification in the conversion of oligodendrocyte precursors to neural stem cells. Genes Dev. 18, 2963-2972 (2004)). The following antibodies were used for detecting antigens: mouse anti-GFAP antibody (1:500, Chemicon Co.), rat anti-nestin antibody (1:1000; BD Pharmingen Co.), mouse anti-MAP2 antibody (a+b) (1:200, Abcam Co.), mouse A2B5 (1:2, supernatant of hybridoma, Developmental Studies Hybridoma Bank), mouse anti-galactocerebroside (GC) antibody (1:2, supernatant of hybridoma, Developmental Studies Hybridoma Bank), rat anti-GFP antibody (1:1000, Nakarai Tesque Co.) and rabbit anti-CD133 antibody (1:50, Abcam Co.). The antibodies were detected using goat anti-rat IgG-A488 (1:400, Molecular Probes Co.), goat anti-mouse IgG-Cy3 (1:400, Jackson ImmunoResearch Co.), goat anti-rabbit IgG-Cy3 (1:400, Jackson ImmunoResearch Co.), goat anti-mouse IgG-A488 (1:400, Molecular Probes Co.) and goat anti-mouse IgM-Texas Red (1:400, Jackson ImmunoResearch Co.). For visualizing all the nuclei, the cells were subjected to counter-staining with 4′,6-diamino-2-phenylindole (DAPI, 1 μg/mL).

Intracranial Cell Transplantation into the Brain of Nude Mouse

Control cells and transfected cells were suspended in 5 μL of the culture medium, and the suspended solution was injected into the brain of a 5 to 8 week old female nude mouse after anesthetizing the animal with 10% pentobarbital. The injection points were stereotactic 2 mm in front of lambdoid suture, 2 mm at the side of sagittal suture and 5 mm in the depth of the brain of the mouse.

### Fixing of the Brain and Histopathology

The incised mouse brain was fixed overnight in 4% paraformaldehyde at 4° C. After fixing, the brain was cryoprotected using a 12 to 18% sucrose solution in PBS, and was embedded in an OCT compound. An annular slice (with a thickness of 10 μm) was prepared from the cerebral cortex, and was stained with hematoxylin-eosin (H & E) using a standard histopathological technique.

### Flow Cytometry

The cells were labeled with prominin 1 (1:200, Abcam Co.), and were analyzed using a dual wavelength analysis (488 nm solid phase laser and 638 nm semiconductor laser) with a JSAN cell sorter (Bay Biosicence Co.). Propidium iodide (PI)-positive cells (i.e. dead cells) were excluded from the analysis.

### Vector Construction

The whole length of mouse HRas was amplified from mouse neural stem cells by RT-PCR by using Phusion polymerase (FINNZYME, Espoo Co.) according to the instruction manual of the manufacturer to clone it in a pDrive vector (Qiagen Co.). HRas^{L61} was obtained by replacing codon 61 glutamine with leucine by PCR. The nucleotide sequence was confirmed using a Big Dye Termination kit (version 3. 1) and an ABI sequencer (model 13130×1). The following oligonucleotide DNA primers were synthesized: (for whole length mouse HRas): 5′-primer; 5′-TGAATTCGCCACCATGACAGAATACAAGCTTGTGTGG-3′ (SEQ. ID NO.: 6), 3′-primer; 5′-ACTCGAGTCAGGACAGCACACATTTGCAG-3′ (SEQ. ID NO.: 7); for Hras^{L61}: 5′-primer; 5′-ACAGCAGGTCTAGAAGAGTATA-3′ (SEQ. ID NO.: 8), 3′-primer; 5′-TATACTCTTCTAGACCTGCTGT-3′ (SEQ. ID NO.: 9).

### Proliferation Assay

2,000 cells were cultivated in each well of a 96-well plate using 100 μL each of a culture medium. The WST-1 assay was implemented as described before for investigating proliferation of the cells (Kawahara, A., et al. Caspase-independent cell killing by Fas-associated protein with death domain. J. Cell Biol. 143, 1353-60 (1998)). A mixture (10 μL) of WST-1 (Dojindo Laboratories) and PMS (Dojindo Laboratories) was added to each well on day 0, day 2 or 3 and day 4 of cultivation. The cells were incubated for 1 hour, and survived cells were quantified by a light absorption spectrum of 595 nm using a micro plate reader (BioRad).

### Soft Agar Assay

A soft agar assay was implemented for investigating whether transfected cells could be proliferated in a scaffold-independent manner. The transfected cells were suspended in 0.3% top agar containing an optimum medium, and the top agar was put on bottom agar containing the same medium. After the top agar is solidified, a culture medium was added onto agar, and the cells were cultivated for 20 days with exchange of the medium every three days. Then, the plate was stained with 0.005% crystal violet for 1 hour at room temperature.

### RT-PCR

RT-PCR was implemented as described before (Kondo, T. & Raff, M. Oligodendrocyte precursor cells reprogrammed to become multipoteintial CNS stem cells. Science 289, 1754-1757 (2000)). Dimethylsulfoxide (DMSO, 5%) was added to a reaction mixture of oligo-1, oligo-2, contactin-1, sox-8 and nestin. The cycle parameters were: 94° C. for 20 seconds, 58° C. for 40 seconds and 72° C. for 45 seconds, and the cycle was repeated 35 times. For gapdh, the cycle parameters were 94° C. for 15 seconds, 53° C. for 30 seconds and 72° C. for 90 seconds, and the cycle was repeated 22 times. The following oligonucleotide DNA primers were synthesized: (for exogenous hras) 5′-primer, 5′-ATGACAGAATACAAGCTTGTGGTG-3′ (SEQ. ID NO.: 10); 3′-primer, 5′-ATTAACCCTCACTAAAGGGAAG-3′ (SEQ. ID NO.: 11): (for s100a6) 5′-primer, 5′-ATGGCATGCCCTCTGGATCAG-3′ (SEQ. ID NO.: 12); 3′-primer, 5′-TTATTTCAGAGCTTCATTGTAGATC-3′ (SEQ. ID NO.: 13); (for prom1), 5′-primer, 5′-AGGCTACTTTGAACATTATCTGCA-3′ (SEQ. ID NO.: 14); 3′-primer, 5′-GGCTTGTCATAACAGGATTGT-3′ (SEQ. IN NO.: 15); (for contactin 1), 5′-primer, 5′-GTCACCAGCCAGGAGTACTC-3′ (SEQ. ID NO.: 16), 3′-primer, 5′-CAGGAGCAAGCTGAGGAGAC-3′ (SEQ. ID NO.: 17); (for mbp) 5′-primer, 5′-ATGGCATCACAGAAGAGACCCT-3′ (SEQ. ID NO.: 18), 3′-primer, 5′-CTGTCTCTTCCTCCCAGCTTAAA-3′ (SEQ. ID NO.: 19); (for plp), 5′-primer, 5′-GACAAGTTTGTGGGCATCACC-3′ (SEQ. ID NO.: 20), 3′-primer, 5′-TCGGCCCATGAGTTTAAGGAC-3′ (SEQ. ID NO.: 21); (for sox8), 5′-primer, 5′-CTATGGAGGCGCTTCCTACTC-3′ (SEQ. ID NO.: 22), 3′-primer, 5′-ACAGGCTGGTCCCAGTTGCT-3′ (SEQ. ID NO.: 23). Primers for nestin, musashi-1, oligo-1, oligo-2 and gapdh were as described before (Kondo, T. & Raff, M. Chromatin remodeling and histone modification in the conversion of oligodendrocyte precursors to neural stem cells. Genes Dev. 18, 2963-2972 (2004) and Wang, S. et al. A role for the helix-loop-helix protein Id2 in the control of oligodendrocyte development. Neuron 29, 603-614 (2001)).

### Microarray Hybridization and Data Processing

Total RNA (5 μg) was subjected to the One-Cycle Target Labeling protocol (Affymetrix Co.) for biotin labeling by in vitro transcription (IVT). Subsequently, cRNA was fragmented, and the fragments were hybridized to the GeneChip Mouse Genome 430 2.0 array (Affimetric Co.) according to the instruction manual of the manufacturer. The microarray image data was processed using a GeneChip Scanner 3000 (Affimetric Co.) to obtain CEL data. Then, the CEL data was analyzed using dChip software (Li, C. & Wong, W. H. Model-based analysis of oligonucleotide arrays: expression index computation and outlier detection Proc. Natl. Acad. Sci. USA, 98, 31-36 (2001)). This permits normalization and processing of multi-data set to be simultaneously performed. The data was normalized in each group according to default setting of the program. Statistical tests were investigated by the eBayes method (Smyth, G. K. Linear models and empirical bayes methods for assessing differential expression in microarray experiments. Stat. Appl. Genet. Mol. Biol. 3, article3 (2004)) and ANOVA with respect to 2- and 3-sample comparison. A q-value as an extension of a value referred to as “FDR (False Discovery Rate)” was investigated as a measure of significance for genome scale tests of significance (Srorey, J. D. A direct approach to false discovery rates. J. R. Satist. Soc. B 64, part3, 479-498 (2002) and Storey, J. D. & Tibshirani, R. Statistical significance for genomewide studies. Proc. Natl. Acad. Sci. USA 100, 9440-9445 (2003)).

### Example 1

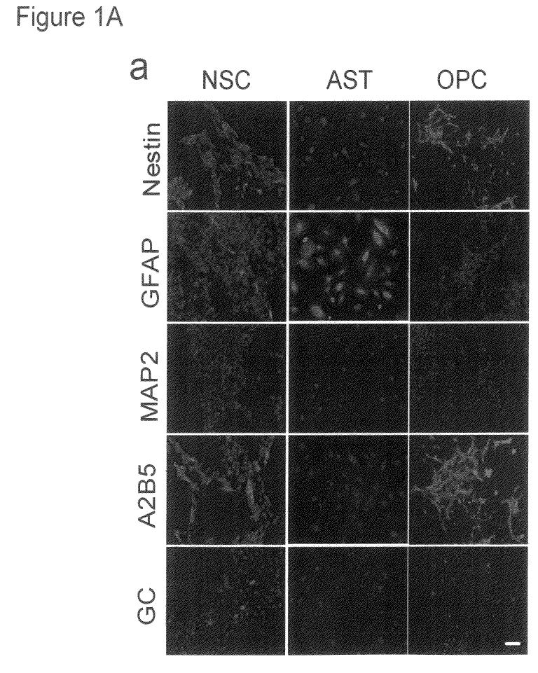

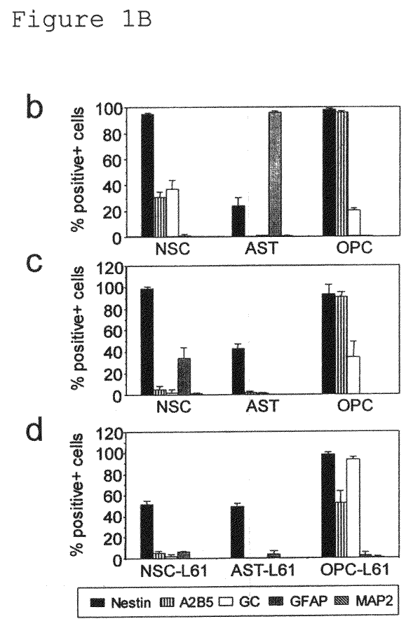

Cells were separated from a p53 (−/−) mouse (Tsukada, T., et al. Enhanced proliferative potential in culture of cells from p53-deficient mice. Oncogene 8, 3313-3322 (1993)) on the embryonic day 13.5 (E13.5), and neuroepithelial cells were expanded in an NSC medium containing bFGF and EGF (Kondo, T. & Raff, M. Chromatin remodeling and histone modification in the conversion of oligodendrocyte precursors to neural stem cells. Genes Dev. 18, 2963-2972 (2004)). 95% or more of the cells were positive to nestin (a well-known NSC marker) under this condition (left side panel in FIG. 1A, and FIG. 1B).

NSCs were cultivated in 10% fatal calf serum (FCS) for 3 weeks or more for inducing proliferation of AST. The result showed that 98% of the cells lost their nestin immunoreactivity, and were immunostained with glia cell fibrous acidic protein (GFAP) as an AST marker (central panel in FIG. 1A, and FIG. 1B).

NSCs were cultivated for 2 weeks or more in the presence of PDGF and in the absence of bFGF and EGF for inducing growth of OPCs, which was immunologically purified using an O4 monoclonal antibody that labels OPCs and oligodendrocytes (Wang, S. et al. A role for the helix-loop-helix protein Id2 in the control of oligodendrocyte development. Neuron 29, 603-614 (2001)). 95% of these cells were A2B5 (a marker of OPCs)-positive, and 98% of the cells were nestin-positive (right panel in FIG. 1A, and FIG. 1B). Differentiation to galactocerebroside (GC) positive oligodendrocyte was induced in 90% or more of the purified OPCs by removing PDGF. This suggests the purity of OPCs.

Subsequently, various cell populations were transfected using either a control vector (which encodes green fluorescence protein (GFP)) or an HRas^{L61} expression vector (which encodes both carcinogenic HRas (Zhu, Y. et al. Early inactivation of p53 tumor suppressor gene cooperating with NF1 loss induces malignant astrocytoma. Cancer Cell 8, 119-130 (2005) and Bizub, D., Blair, D., Alvord, G. & Skalka, A. M. Correlation between H-ras p21TLeu61 protein content and tumorigenicity of NIH3T3 cells. Oncogene 3, 443-448 (1988)) in which glutamine is substituted with leucine in codon 61 and GFP). The transfected cells were cultivated for 2 to 3 weeks, and GFP-positive cells were purified by flow cytometry.

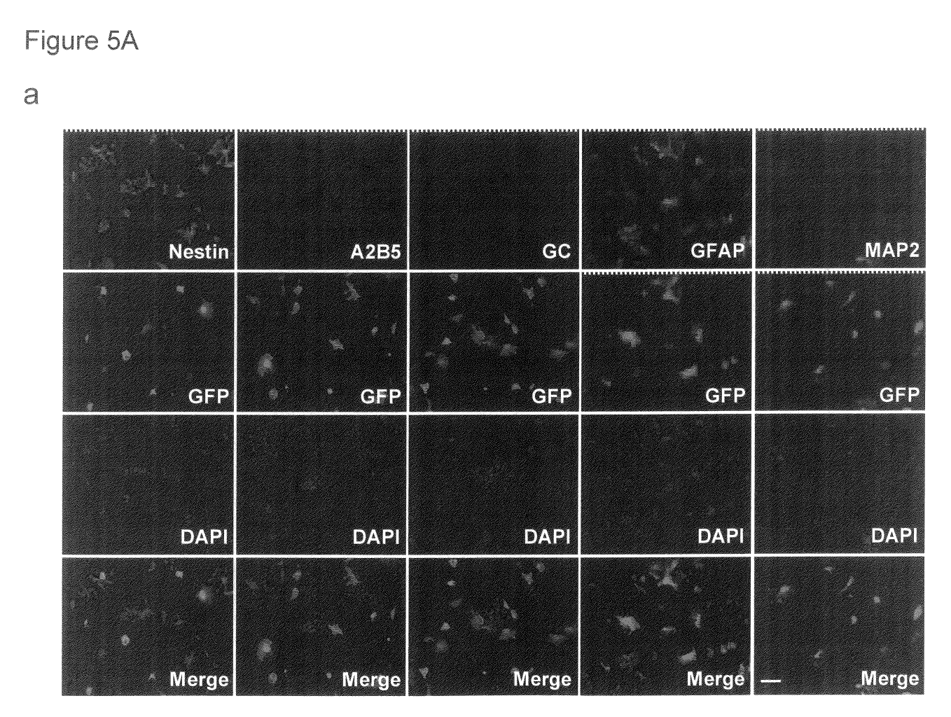

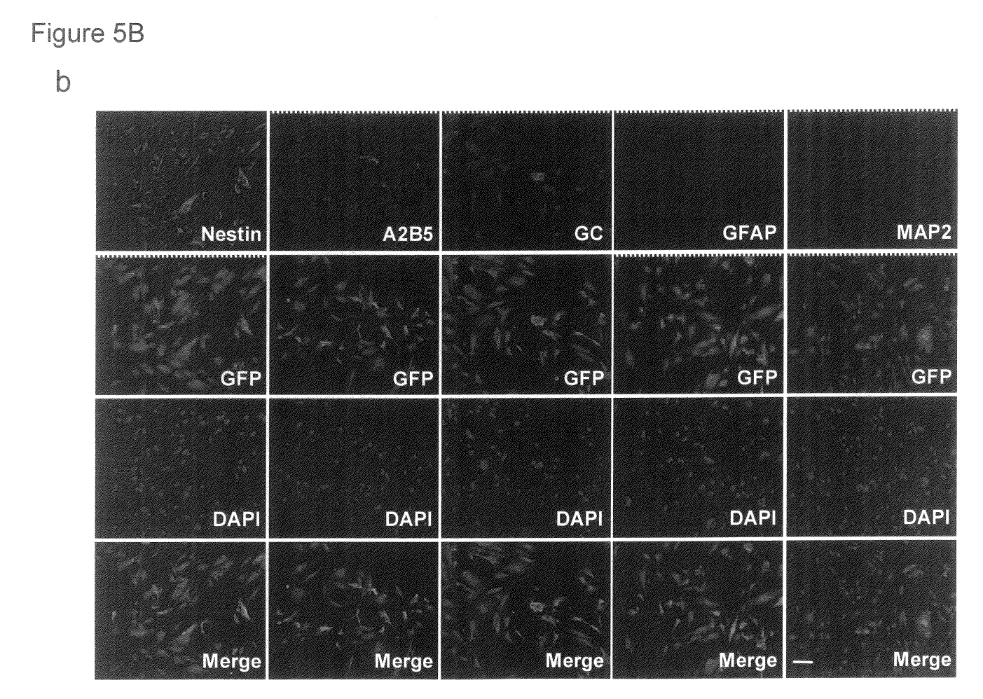

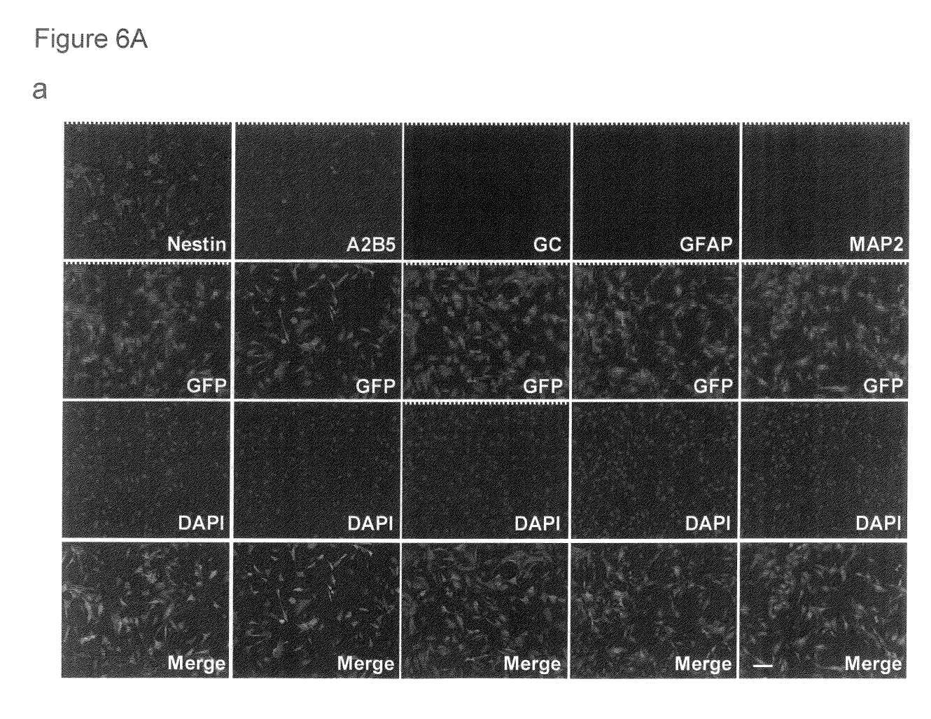

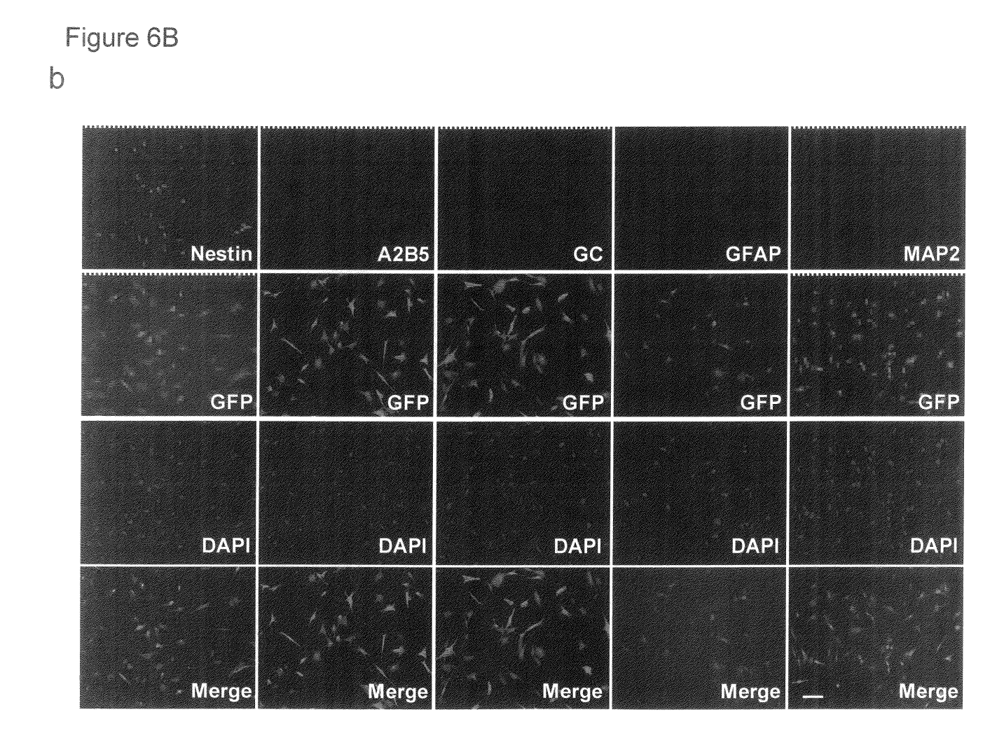

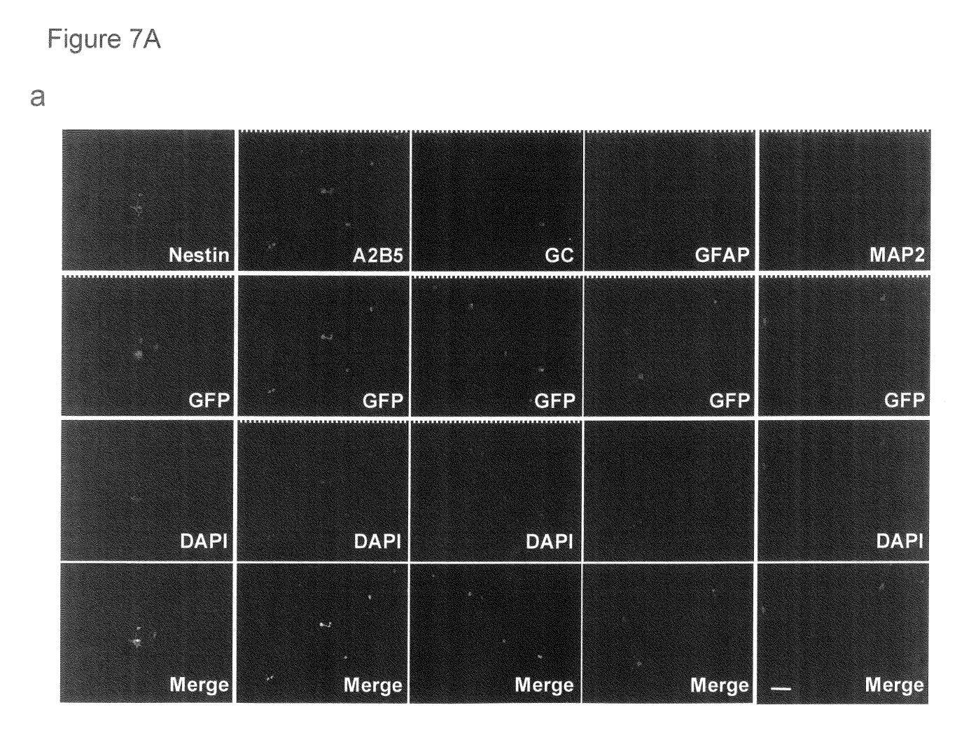

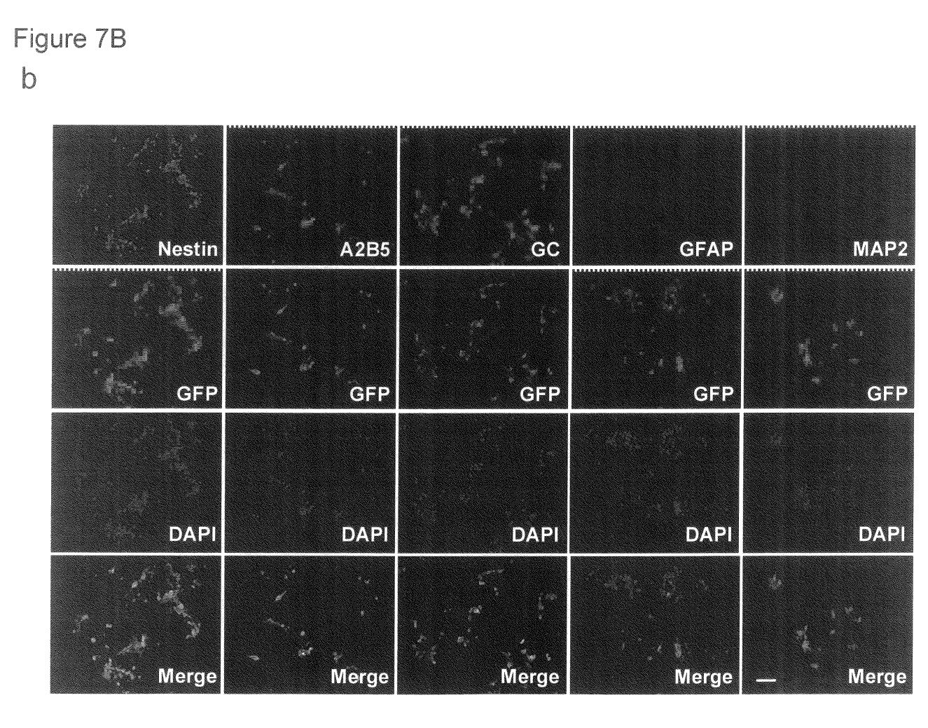

Then, these GFP positive transfected cells were immunolabeled for nestin, A2B5, GFAP, GC and MAP2 (a neuronal marker). 99% of the control vector transfect NSC(NSC-C) and 51% of the HRas^{L61} vector transfect NSC (NSC-L61) were labeled with nestin. 6% of NSC-C and 5% of NSC-L61 were positive to A2B5. While 34% of NSC-C was also labeled with GFAP, only less than 5% of NSC-L61 was labeled with GFAP. Less than 3% of NSC-C and NSC-L61 were positive to GC or MAP2 (FIGS. 1C and 1D, and FIG. 5). While control vector transfect AST (AST-C) or HRas^{L61} vector transfect AST (AST-L61) lost GFAP immunoreactivity when cultivated in 10% FCS, representative morphological features of the differentiated astrocyte were maintained (FIGS. 1C and 1D, and FIG. 6). This fact coincides with the former knowledge that loss of GFAP expression in p53 (−/−) astrocyte advances during subculture in FCS (Bogler, O., Huang, H. J. & Cavenee, W. K. Loss of wild-type p53 bestows a growth advantage on primary cortical astrocytes and facilitates their in vitro transformation. Cancer Res. 55, 2746-2751 (1995)). In addition, while 43% of AST-C and 50% of AST-L61 were labeled with nestin, they were not positive to any other marker. 98% or more of the control vector transfect OPC (OPC-C) or HRas^{L61} vector transfect OPC(OPC-L61) was nestin-positive (FIGS. 1C and 1D, and FIG. 7). 95% and 35% of OPC-C were labeled with A2B5 and GC, respectively, and 51% and 90% of OPC-L61 were A2B5-positive and GC-positive, respectively. OPC-C or OPC-L61 was not labeled with any other marker. Transfected oligodendrocyte was not proliferated, and was extinct within two weeks. These data show that OPC-L61 maintains its intrinsic phenotype, while NSC-L61 and AST-L61 partially lost expression of their specific antigen markers.

### Example 2

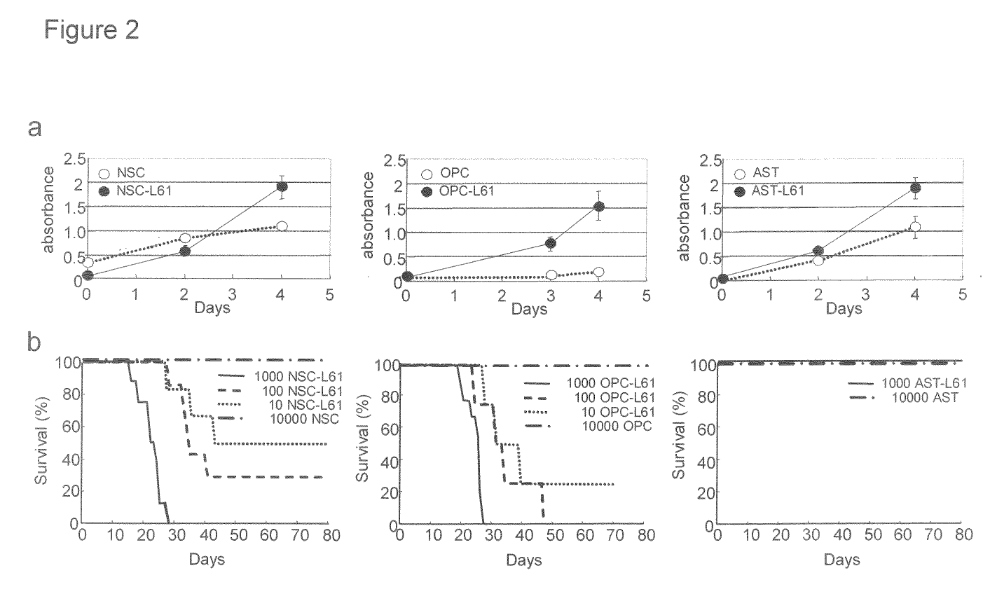

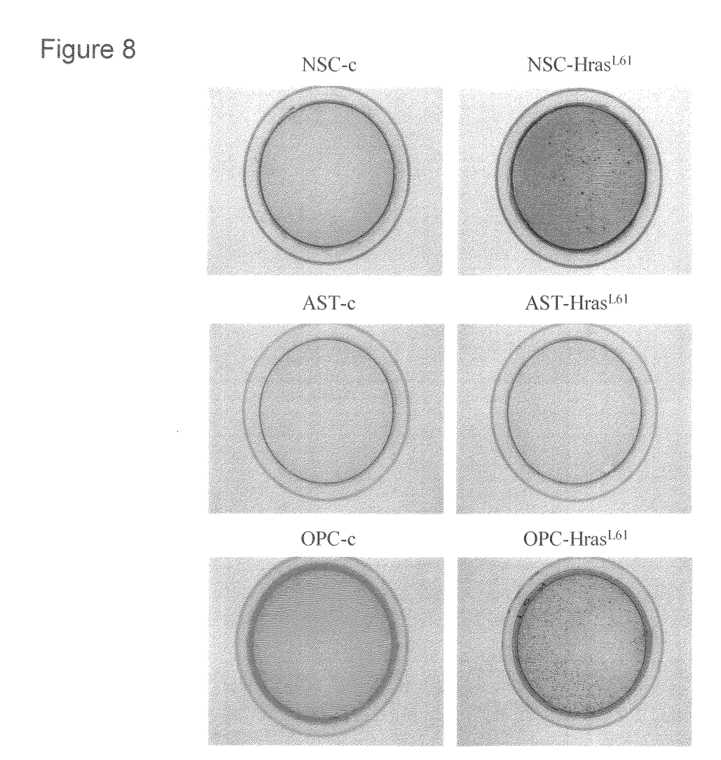

Next, we have investigated whether activation of Ras could induce transformation of NSCs, ASTs or OPCs. It was found that activation of Ras significantly enhances proliferation of all the transfected cells by using the WST-1 assay for measuring proliferation and the survival rate of the cells (FIG. 2A). It was also found that both NSC-L61 and OPC-L61 form colonies in soft agar, but AST-L61 does not form any colony (FIG. 8). These results suggest that, while a combination of Ras activation and p53 deficiency could arise transformation of NSCs and OPCs, ASTs are not transformed.

Transfected cells (10 to 1,000 cells) were injected into the brain of a nude mouse for investigating the tumor-forming ability of the transfected cell and the frequency of CSC. All the mice that received either 1,000 cells of NSC-L61 or 1,000 cells of OPC-L61 died of brain tumor within 30 days, while the mice that received either 1,000 cells of AST-L61, 10⁵ cells of NSC-C, 10⁵ cells of AST-C or 10⁵ cells of OPC-C showed no evidence of tumorigenesis in the brain (FIG. 2B). Brain tumor was formed in 5 of 7 mice that received 100 cells of NSC-L61 and in 3 of 6 mice that received 10 cells of NSC-L61. This shows one of about 20 cells of NSC-L61 is CSC (left side of FIG. 2B). In the case of OPC-L61, all the mice (5 of 5 mice) that received 100 cells and 3 of 4 mice that received 10 cells suffered from brain tumor and died. This suggests that 3 of 40 cells of OPC-L61 are CSC (center in FIG. 2B).

### Example 3

Histopathology of a brain tumor was investigated for determining the nature of the brain tumor induced from NSC-L61 and OPC-L61. The brain was fixed, and frozen slices were stained with hematoxylin and eosin (H & E), or with GFP for identifying transfected cells.

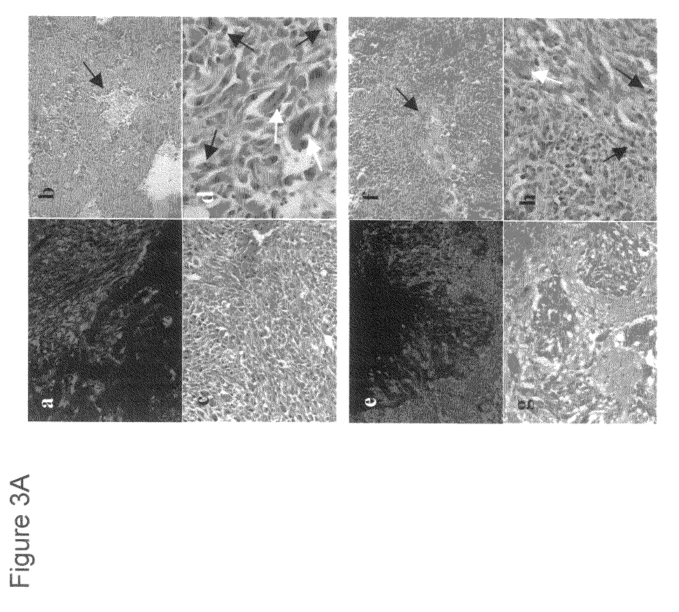

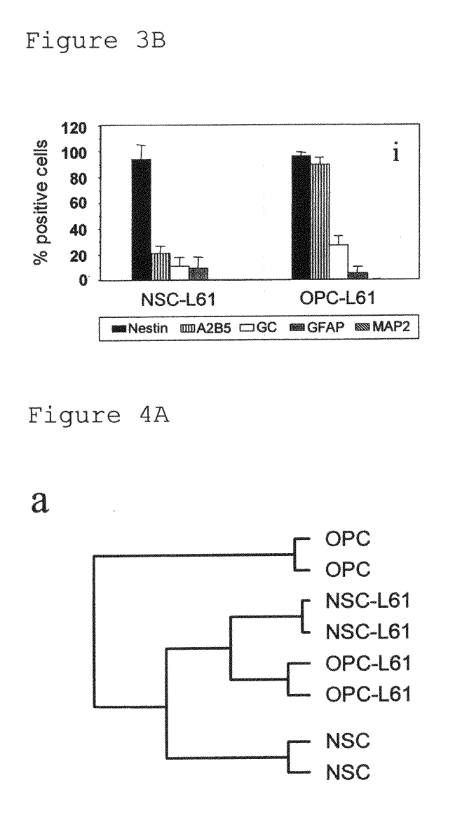

As shown in FIGS. 3A and 3E, the tumor was composed of GFP-positive cells, and most of them seemed to be an adjoining infiltrated brain tissue. H & E staining revealed that these tumors showed the feature of polymorphic human glioblastoma (GBM) (including hypercellularity, polymorphism, multinucleated giant cells, mitosis, necrosis and infiltration into adjoining brain tissues) (FIGS. 3B to 3D, 3F to 3H).

For further characterizing the tumor, it was isolated and dispersed cells were immediately immunolabeled with both GFP and a neuro-marker (FIG. 3I). In the tumor formed by NSC-L61, 95% and 20% of GFP-positive cells were nestin-positive and A2B5-positive, respectively, while less than 10% of GFP-positive cells was GFAP-positive or GC-positive. In the tumor formed by OPC-L61, 95% and 93% of GFP-positive cells were nestin-positive and A2B5-positive, respectively. While 25% of the cells was also GC-positive, less than 5% was GFAP-positive. No MAP2-positive cells were found in any of these tumors. Accordingly, it was suggested that the tumors formed from NSC-L61 and OPC-L61 contain a mixture of cells that express markers for the oligodendrocyte and astrocyte.

### Example 4

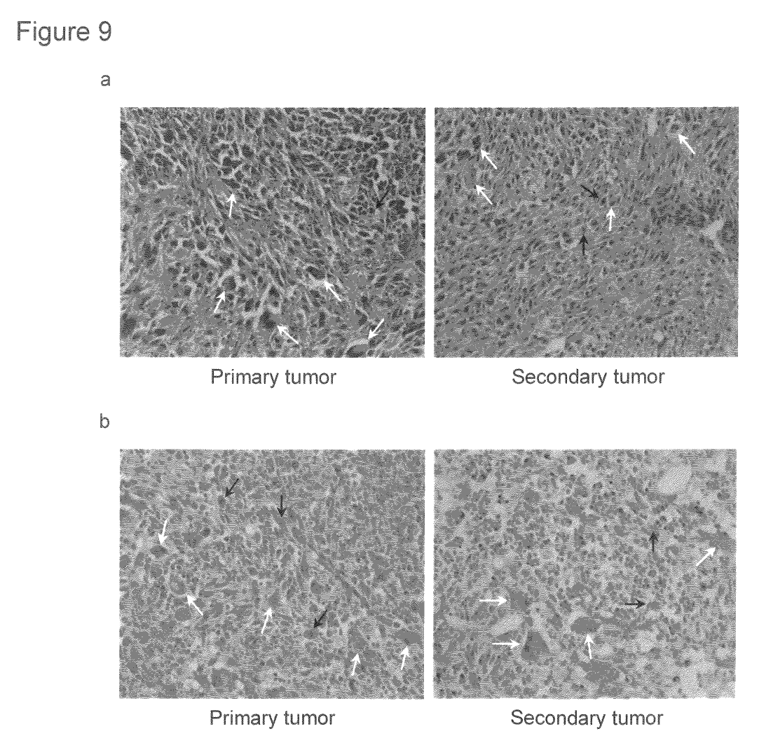

The principal feature of all the stem cells including CSC is the infinite self-regenerative ability. For determining whether NSC-L61 and OPC-L61 have this ability, a continuous transplantation experiment was performed using cells originated from tumors formed by either 100 cells of NSC-L61 or 100 cells of OPC-L61. Primary tumors were excised from the brain of a mouse, 100 cells of GFP-positive cells were purified by flow cytometry, and the purified cells were re-injected into a second mouse. After 4 weeks, 3 of each 3 of these mice formed a brain tumor quite close to the phenotype of the primary tumor (FIG. 9). This shows the in-vivo self-regenerative ability of NSC-L61 and OPC-L61. These data suggests that both NSC-L61 and OPC-L61 contain glioma stem cells in a substantial proportion.

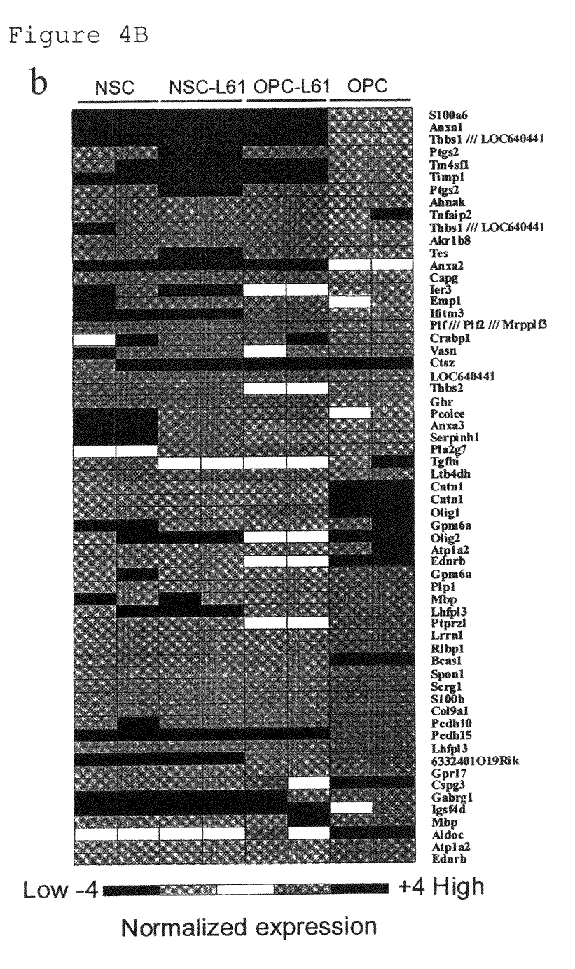

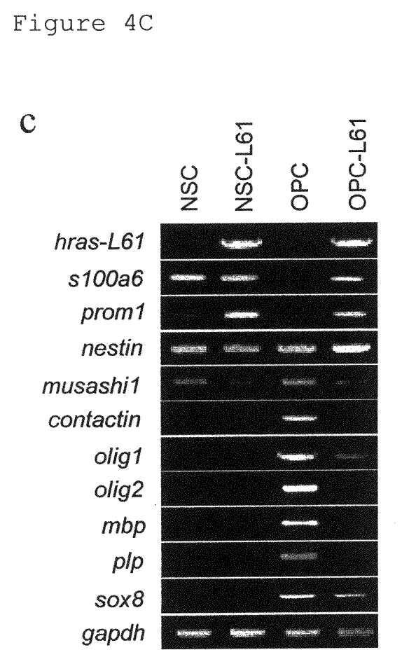

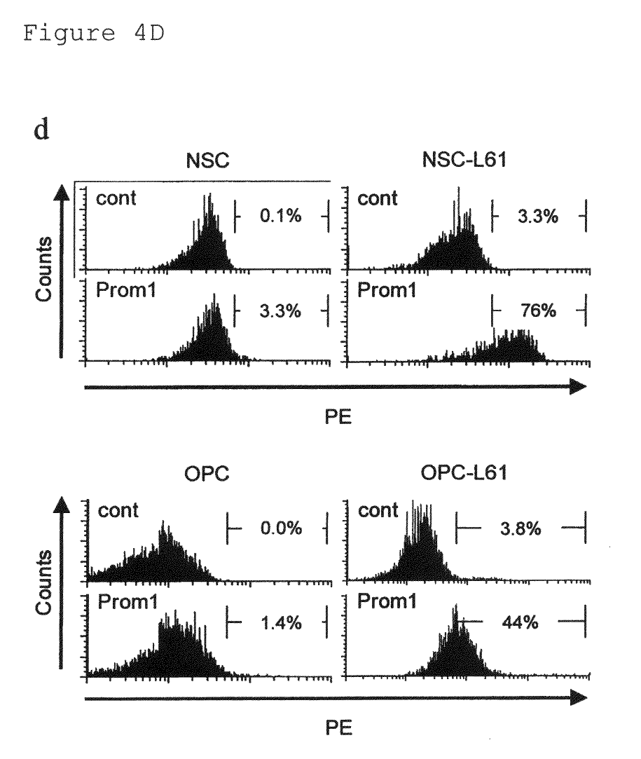

Genes expression profiling were used for characterizing the change of gene expression occurred during transformation of NSC and OPC. Total RNAs were purified from NSC, OPC, NSC-L61 and OPC-L61, the purified RNAs were amplified, and the amplified RNAs were hybridized on Affimetrix Murine 430A 20 microarray (Affimetrix Co.). The result of analysis showed that, while the gene expression profile of OPC-L61 resembles those of both NSC and NSC-L61, it is different from the profile of OPC (FIG. 4A). A decrease in expression in OPC-L61, and vice versa, of a group of genes that are highly expressed in OPC was also shown. This suggests that OPC have experienced collective re-programming of gene expression during transformation to OPC-L61 (FIG. 4B). Subsequently, RT-PCR was applied to determine whether OPC-L61 acquired expression of NSC specific genes and lost expression of OPC specific genes. As shown in FIG. 4C, prominin 1 (prom-1) and s100a6 as stem cell markers were expressed in OPC-L61, but were not expressed in OPC. In contrast, OPC-L61 lost expression of OPC specific genes (contactin/F3, olig2, myelin basic protein (mbp) and proteolipid protein (plp)). Expression of oligo-1 and sox-8 (these are also OPC series genes) was significantly decreased in OPC-L61. These results suggest that OPC had acquired expression of NSC-specific genes and lost expression of OPC-specific genes during transformation to OPC-L61.

Much evidence has been accumulated on enrichment of brain CSC in CD133 (human Prom1)-positive cancer cells originated from gliomas and medulloblastoma (Singh, S. K. et al. Identification of human brain tumour initiating cells. Nature 432, 396-401 (2004); Galli, R. et al. Isolation and characterization of tumorigenic, stem-like neural precursors from human glioblastoma. Cancer Res 64, 7011-7021 (2004); Bao, S. et al. Glioma stem cells promote radioresistance by preferential activation of the DNA damage response. Nature 444, 756-760 (2006); Piccirillo, S. G. et al. Bone morphogenetic proteins inhibit the tumorigenic potential of human brain tumour-initiating cells. Nature 444, 761-765 (2006); and Hambardzumyan, D., Squatrito, M. & Holland, E. C. Radiation resistance and stem-like cells in brain tumors. Cancer Cell 10, 454-456 (2006)). Since both NSC-L61 and OPC-L61 highly express prom1 mRNAs (FIG. 4C), the frequency of Prom1-positive cells in these transformants were evaluated. NSC, OPC, NSC-L61 and OPC-L61 were labeled with Prom1, and the labeled cells were analyzed by flow cytometry. 76% of NSC-L61 and 44% of OPC-L61 were Prom1-positive, while only 3% of NSC and 1% of OPC were Prom1-positive (FIG. 4D). This coincides with the former results that Prom1 is highly expressed in CSC.

The present inventors have verified that both NSC and OPC could be transformed to glioblastoma cells with collective changes of gene expression by the combination of Ras activation and p53 deficiency. The results by the present inventors suggest that the oligodendrocyte progenitor cell (OPC) having specified oncogenic mutation may be transformed to CSC. This suggests a possibility that at least some of human glioblastoma may originate from OPC, which coincides with the recent results by Ligon et al. (Ligon, K. L. et al. Olig2-Regulated Lineage-Restricted Pathway Controls Replication Competence in Neural Stem Cells and Malignant Glioma. Neuron 53, 503-517 (2007)). The main challenge at present is to clearly identify CSC in human glioblastoma in order to find a way for extinguishing it.

The present invention provides a method for preparing cancer stem cells; the cancer stem cells prepared by the preparation method; a method for screening a cancer stem cell-targeting substance and a method for screening an anti-cancer substance using the cancer stem cells; pharmaceutical compositions containing the substances obtainable by the screening methods; and a diagnostic method for cancers including the step of detecting proteins specifically expressed in the cancer stem cells or mRNAs of the protein.

1. A method for preparing cancer stem cells, comprising the step of subjecting normal cells to Ras activation and p53 deficiency.

2. The method according to claim 1, wherein the cancer stern cells are glioma stem cells.

3. The method according to claim 1, wherein the normal cells are neuronal stem cells or oligodendrocyte progenitor cells.

4. The method according to claim 1, wherein Ras activation is achieved by introduction of an HRas^{L61} gene into the neuronal cells.

5. The method according to claim 1, wherein p53 deficiency is achieved by using cells originated from a p53 deficient animal.

6. Cancer stem cells obtainable by the method according to of claim 1.

7. A method for screening a cancer stem cell-targeting substance, comprising the step of identifying the cancer stem cell-targeting substance by determining molecules specifically expressed in the cancer stem cells according to claim 6.

8. A method for treating cancer in a patient, comprising the step of administering to said patient the cancer stem cell-targeting substance obtainable by the method according to claim 7.

9. A method for screening an anti-cancer substance, comprising the step of adding a candidate substance to the cancer stem cells according to claim 6.

10. A method for treating cancer in a patient, comprising the step of administering to said patient the cancer stem cell-targeting substance obtainable by the method according to claim 9.

11. A method for diagnosing cancers, comprising the step of detecting proteins or mRNAs thereof specifically expressed in the cancer stem cells according to claim 6.
